(19) United States
(12) Patent Application Publication
(10) Pub. No.: US 2025/0058037 A1
(43) Pub. Date: Feb. 20, 2025

(54) Device for Administering Fluid Into an Orifice of a Human or an Animal

(71) Applicants: Joseph Richard Cant, Naples, FL (US); Gary Michael Wilson, Naples, FL (US)

(72) Inventors: Joseph Richard Cant, Naples, FL (US); Gary Michael Wilson, Naples, FL (US)

(21) Appl. No.: 18/452,183

(22) Filed: Aug. 18, 2023

(51) Int. Cl. A61M 3/02

(52) U.S. Cl. CPC A61M 3/0279

(57) ABSTRACT

A device for administering a fluid into a human cavity or animal cavity through an orifice includes a container for housing a fluid. The container includes a bottom portion, a tip portion, and a directional flange disposed between the bottom portion and the tip portion. The bottom portion has a bottom, a squeezing section, and a narrowing section adjacent the directional flange. The tip portion has an end section with an end face and a body section defining an elongated structure extending from the directional flange to the end face. The directional flange comprises an upper surface and a lower surface connected to one another by an edge and the edge has a rigidity less than the tip portion such that the edge collapses in response to a force applied to the tip portion causing movement of the end face relative to the bottom portion.

### BACKGROUND

### Field of the Invention

The subject invention relates to a device for administering fluid through a nozzle that articulates relative to an orifice of a human or animal creating a seal against the orifice to deliver a jet of fluid to cleanse, medicate, stimulate evacuation, or for diagnostic purposes, and the like such that the device has a safety flex neck.

### Description of Related Art

An enema is a common medical procedure whereby fluid is injected into a rectum of a patient to induce bowel movement. The need for such a procedure typically arises in patients suffering from certain physical ailments in which voluntary bowel control is impaired. At least one type of device or enema used for such a procedure includes squeeze bottle

filled with the fluid intended to induce bowel movement. The squeeze bottle is capped by an applicator nozzle configured to be inserted into the patient's rectum through the anal opening or anus. The applicator nozzle of this type of the related art enema device often causes discomfort and irritation when being inserted.

Disposable enemas for self-application include an enema squeeze bottle filled with the fluid intended to induce bowel movement. This bottle is capped by a long pointed anal insertion fluid delivery nozzle. The nozzle tip is configured to penetrate the anal opening and to be inserted past the exterior and interior sphincter muscles. The fluid filled bottle is squeezed causing the fluid to be jettisoned into the rectal cavity.

The nozzle of the related art disposable enema application devices often causes discomfort and irritation when inserted into the rectum. Extreme care must be taken during the insertion process to avoid injuring the delicate rectal tissue or puncturing existing polyps or hemorrhoids.

Another such device used for these types of procedures is disclosed in U.S. Pat. No. 9,227,006. The device includes a non-penetrating nozzle and a squeezable body with a fixed connection therebetween. Although the non-penetrating nozzle provides an adequate seal with the orifice, the device provides no flexibility or adjustability between the placement of the nozzle and the body of the device. Because the area of application and use for such device is relatively limited, it can be difficult to maintain the proper seal while also ejecting the fluid.

Therefore, there is a need for a device, and a method of using the device, that delivers fluid into the orifice by a nozzle that provides additional flexibility and accommodations to the user while dispensing the fluid therefrom.

### SUMMARY

A device for administering a fluid into a human cavity or animal cavity through an

orifice is provided that includes a container for housing a fluid. The container includes a bottom portion, a tip portion, and a directional flange disposed between the bottom portion and the tip portion. The bottom portion has a bottom, a squeezing section, and a narrowing section adjacent the directional flange. The tip portion has an end section with an end face and a body section defining an elongated structure extending from the directional flange to the end face. The directional flange comprises an upper surface and a lower surface connected to one another by an edge and the edge has a rigidity less than the tip portion such that the edge collapses in response to a force applied to the tip portion causing movement of the end face relative to the bottom portion.

The subject invention provides the device with a safety flex neck to deliver fluid into the rectal cavity through the nozzle while providing additional flexibility between the nozzle and the device to achieve the desired seal with the orifice and a more comfortable position of the bottom portion for the user to actuate and deliver the fluid. Moreover, as the squeezing section is squeezed and fluid is ejected, because the alignment and positioning of the nozzle relative to the bottom portion is flexible, continued squeezing can occur even in spite of the limited space for application.

### BRIEF DESCRIPTION OF THE DRAWINGS

Other advantages of the present invention will be readily appreciated as the same becomes better understood by reference to the following detailed description when considered in connection with the accompanying drawings wherein:

FIG. 1 is a side view of one embodiment of a device for administering a fluid into a human cavity or animal cavity;

FIG. 2 is a cross-sectional view of the device shown in FIG. 1;

FIG. 3 is a side view of another embodiment of the device for administering a fluid into a human cavity or animal cavity;

FIG. 4 is a cross-sectional view of the device shown in FIG. 3;

FIG. 5 is a side view of the device shown in FIG. 3 having a nozzle separated therefrom;

FIG. 6 is a cross-sectional view of the device shown in FIG. 3 subjected to an orthogonal force;

FIG. 7 is a side view of the device shown in FIG. 3 subjected to a transverse force;

FIG. 8 is a cross-sectional view of the device shown in FIG. 3 subjected to a transverse force;

FIGS. 9 is a side view of the device shown in FIG. 3 subjected to a transverse force;

FIG. 10 is a cross-sectional view of the device shown in FIG. 3 subjected to a transverse force; and

FIG. 11 is a schematic representation of the device adjacent a cavity administering a fluid therefrom.

### DETAILED DESCRIPTION

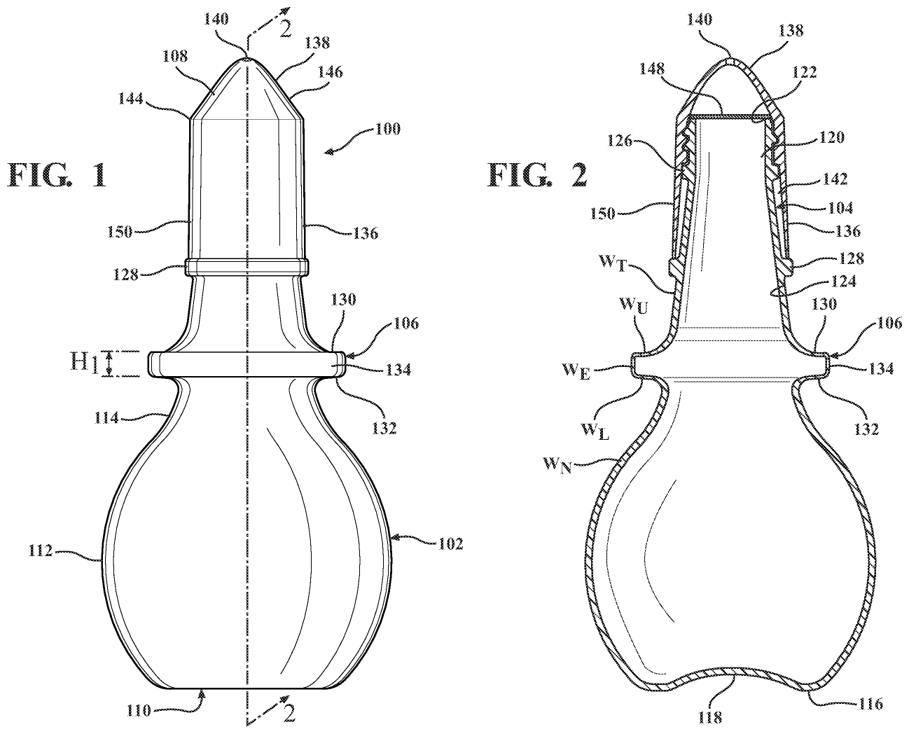

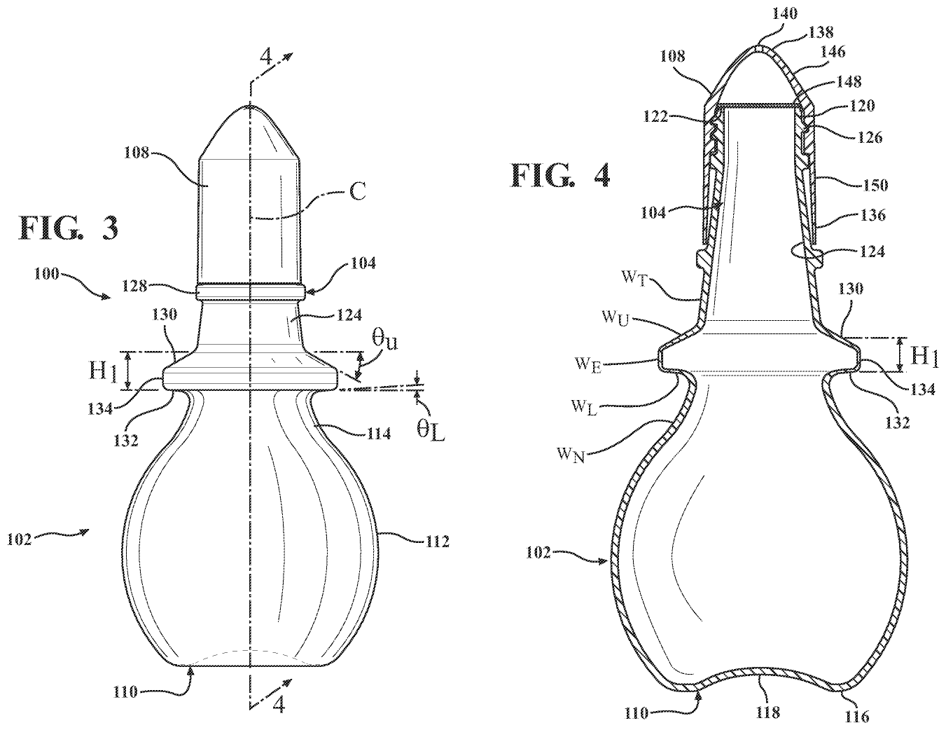

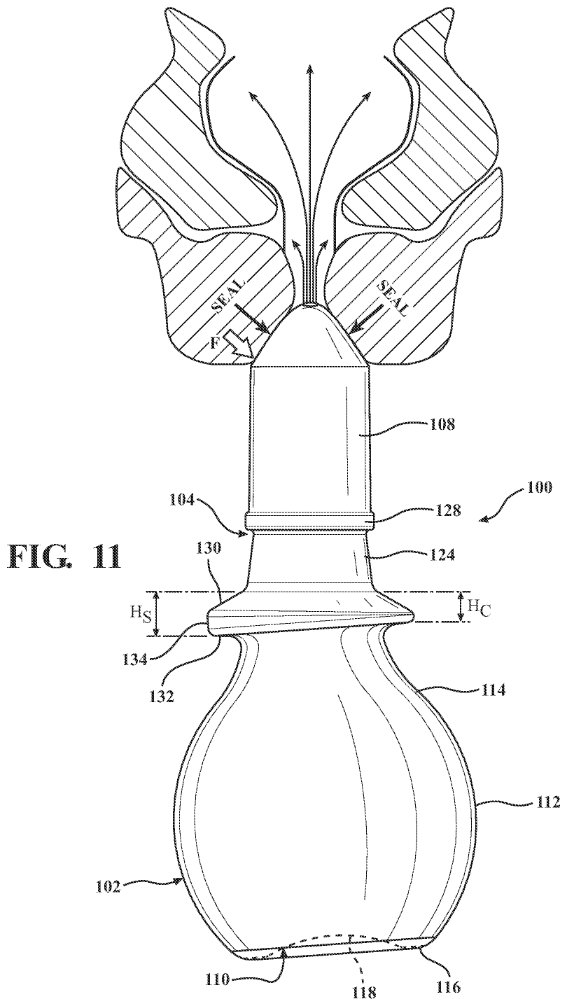

Referring to the Figures, wherein like numerals indicate like or corresponding parts throughout the several views, one embodiment of a device for administering a fluid into a human cavity or animal cavity through an orifice is shown generally at 100 in FIGS. 1 and 2. Another embodiment of the device 100 is shown in FIGS. 3-11. It is to be appreciated that like numerals throughout the several Figures refer to the same element. The device 100 may be used by a person to self-administer the enema or another fluid, such as the fluid for an enema or a douche. The device 100 includes a container that includes a bottom portion 102, a tip portion 104, and a directional flange 106 disposed between the bottom portion 102 and the tip portion 104. The bottom portion 102 generally houses the fluid before it is administered. The device 100 also includes a nozzle 108 coupled to the container for delivering the fluid from the container into the human cavity or the animal cavity through the orifice. Exemplary embodiments provide a flow-through enema nozzle that does not penetrate the anus or does not pass the exterior or interior sphincter muscles, but instead is pressed against the anal opening while it delivers a jet of liquid from the liquid filled squeeze bottle into the anus and rectal cavity for cleansing, stimulating evacuation, or for diagnostic purposes.

The bottom portion 102 includes a bottom 110, a squeezing section 112, and a narrowing section 114 adjacent the directional flange 106. The bottom 110 is shown as having a partial flat surface 116 for supporting the device 100 on a surface. The bottom 110 also includes a concave center 118 that extends upward from the partial flat surface 116. The squeezing section 112 is shown as having a generally bulbous shape. The squeezing section 112 would be grasped by a user and squeezed to eject the fluid from the container. Exemplary embodiments also provide for any shaped container to deliver an appropriate amount of prescribed liquid from an attached liquid-filled squeeze bottle, squeeze bulb of any shape or capacity or any appropriate fluid container into the anus and rectal cavity and/or vagina for cleansing, pre-sexual penetration lubrication, pre-bowel movement rectal and/or hemorrhoid lubrication, rectal delivery of medication or laxatives, to stimulate evacuation, or for pre or post-diagnostic purposes. For purposes of clarity, the following description is made primarily in the context of an enema application in accordance with the invention, however, the same inventive features may be used as a means of a non-penetrating fluid delivery nozzle into the vagina or uterine cavity as well.

The tip portion 104 has an end section 120 with an end face 122 and a body section 124 defining an elongated structure extending from the directional flange 106 to the end face 122. The tip portion 104 may also be referred to as a neck. The tip portion 104 has a conical shape and tapers towards the end face 122. The tip portion 104 may include threads 126 for threadably engaging the nozzle 108 with a threaded connection. A cap (not shown) may also be included which covers the nozzle 108. The tip portion 104 further includes an abutment flange 128 disposed about the tip for abutting the nozzle 108 when the nozzle 108 is connected to the tip portion 104.

The directional flange 106 includes an upper surface 130 and a lower surface 132 connected to one another by an edge 134. The edge 134 has a rigidity less than the tip portion 104 such that the edge 134 collapses in response to a force applied to the tip portion 104 causing movement of the end face 122 relative to the bottom portion 102. Said differently, the tip portion 104, and specifically, the end face 122 is able to be aligned with the orifice and the bottom portion 102 can be angled or positioned for the convenience of the user because the directional flange 106 can compress or pivot as needed. The edge 134 has a wall thickness, W_{E}, that defines the rigidity. It is to be appreciated that the wall thickness and rigidity may also be described as the edge 134 being flexible or deformable, which are further defined by the wall thickness and rigidity of the material forming the device 100. Alternatively, the rigidity could be determined by specific types of material or a manner of forming the device 100, and specifically, how the directional flange 106 is formed. One type of material useful in forming the device 100 is a plastic material and formed in an injection molding process. One of ordinary skill may use other materials and methods, while still practicing the subject invention.

In the embodiment shown in FIG. 1, the upper and lower surfaces 130, 132 are generally flat. The upper surface 130 is radiused into the body of the tip portion 104 and the lower surface 132 is radiused into the narrowing section 114 of the bottom portion 102. In order to allow for additional alignment of the end face 122 relative to the orifice, the narrowing section 114 of the bottom portion 102 may also have a rigidity that is more than the edge 134. In other words, the narrowing section 114 is stiffer than the edge 134 so as the device 100 is manipulated, any forces or directions applied to the bottom portion 102 further assist with the alignment of the end face 122. In certain embodiment, the rigidity of the upper and lower surfaces 130, 132 are the same and are greater than the rigidity of the edge 134. Additionally, the rigidity of the upper and lower surfaces 130, 132 may be different, so long as at least one is more rigid than the edge 134 to act as a force transmitting section to the edge 134.

As described above, wall thickness is one way to set the rigidity of the material. Referring to FIG. 2, a cross-sectional view of the embodiment of FIG. 1 is shown. The edge 134 has the wall thickness WE that is less than a wall thickness W_{T} of the body section 124 of the tip portion 104 for allowing movement therebetween when the tip portion 104 is subjected to a force. In certain embodiments, the upper surface 130 may have a wall thickness W_{U} that is the same as the edge 134 thickness W_{E} and the lower surface 132 may have a wall thickness W_{L} that is the same as the edge 134 thickness W_{E}. In other embodiments, the upper surface 130 has the wall thickness W_{U} that is greater than as the wall thickness W_{E} of the edge 134, or the lower surface 132 has the wall thickness W_{L} that is greater than as the wall thickness W_{E} of the edge 134. Alternatively, the wall thicknesses W_{U}, W_{L} of the upper surface 130 and the lower surface 132 may be the same.

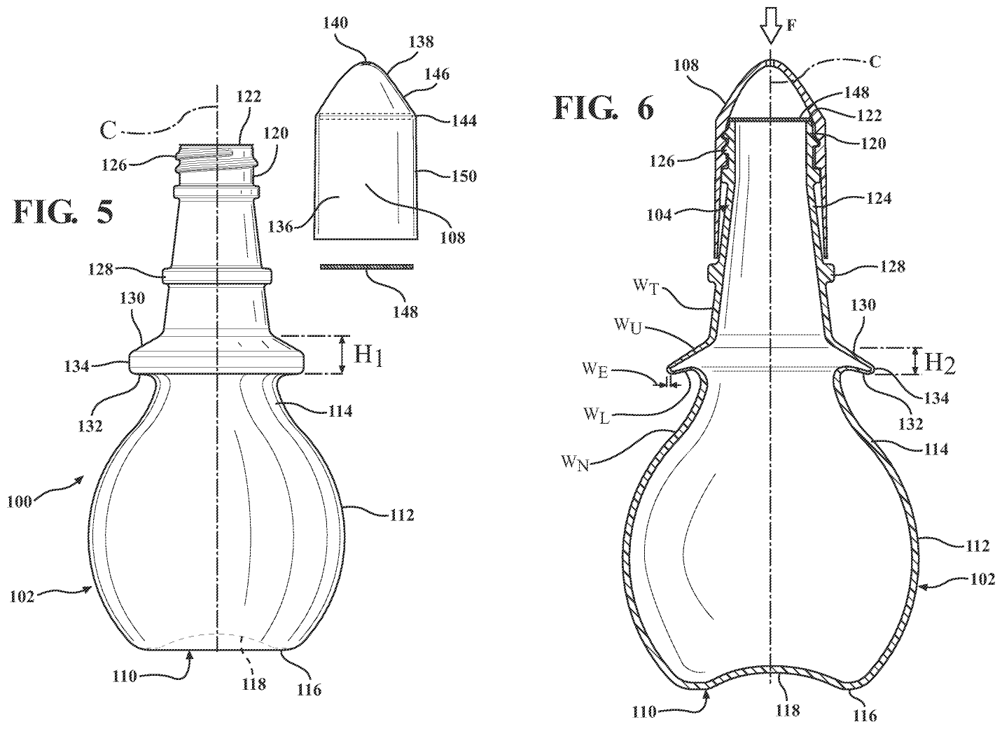

FIG. 3-4 show another embodiment of the device 100 and FIG. 5 shows the nozzle 108 removed from the tip portion 104. In the embodiment shown, the upper surface 130 has an inclined upper angle θ_{U} greater than zero degrees and less than 55 degrees, preferably less than 45 degrees. The lower surface 132 may be a recessed surface with an inclined lower angle θ_{L} of less than 20 degrees. In the embodiment shown, the lower surface 132 is substantially flat, surface with an inclined angle of less than 5 degrees, preferably less than 3 degrees. The edge 134 has the wall thickness W_{E} that is less than the wall thickness W_{R} of the body section 124 of the tip portion 104 and less than the wall thickness of the narrowing section 114. The wall thickness Wu of the upper surface 130 is the same as the wall thickness W_{L} of the lower surface 132.

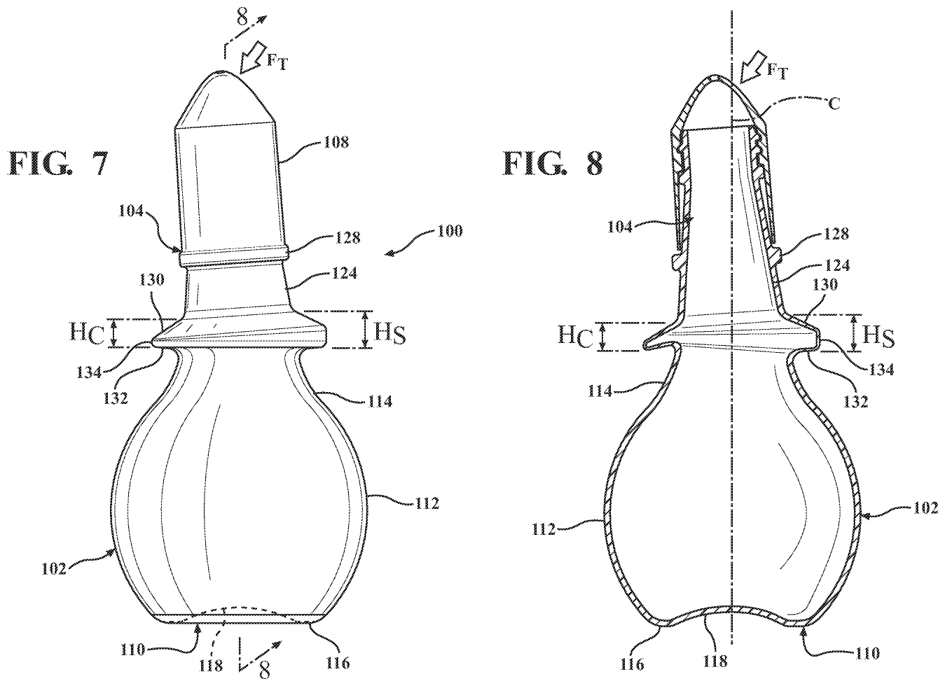

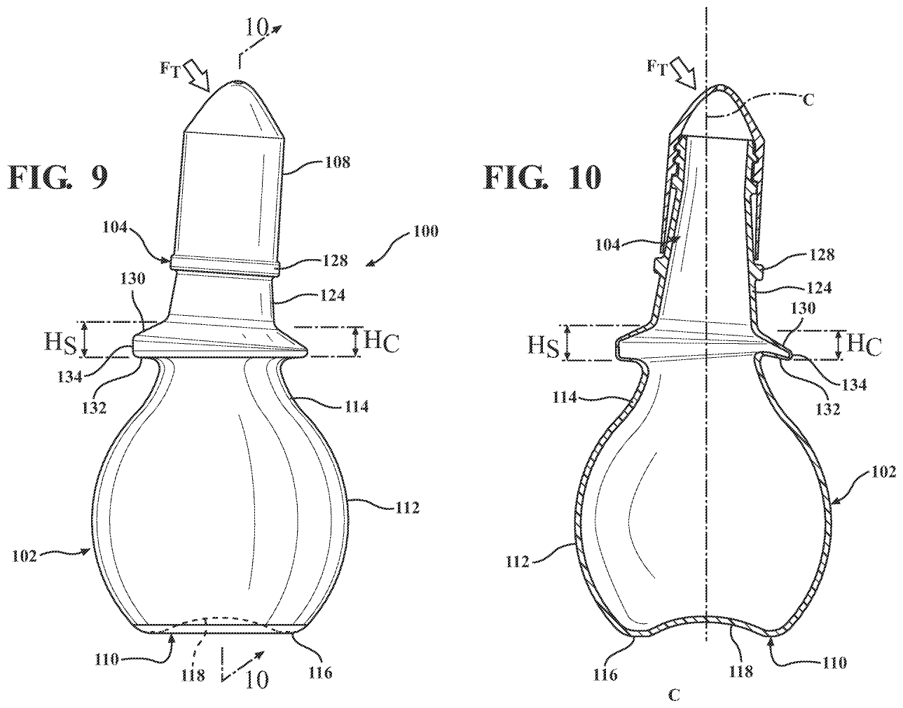

Generally, the directional flange 106 has a first height H₁ when no forces are applied to the tip portion 104. FIGS. 1 and 3 each show the device 100 under no external forces so the directional flange 106 has the first height H₁. With reference to FIG. 6, an orthogonal force, F, is applied to the tip portion 104, the directional flange 106 compresses to a second height H₂. The orthogonal force, F, is being applied along a longitudinal, central axis C of the device 100. If a force transverse to the longitudinal axis C, i.e. transverse force, F_{T}, is applied to the tip portion 104, then one side of the directional flange 106 is compressed and has a compressed height H_{C} and the other side has a stretched height H_{S}. Referring to FIGS. 7-8, the transverse force F_{T} is applied from the right of the central axis C, so that the left side of the directional flange 106 is compressed and has the compressed height H_{C}. The opposite, left side is then stretched and has the stretched height H_{S}. FIGS. 9-11 shows the transverse force F_{T} applied from a left side of the device 100, while the device 100 is making a seal with the cavity. It is to be appreciated that the force being applied to the device 100 results in the alignment of the nozzle 108 with the orifice, and as such, the force may be shown as a counterforce. Referring back to the cross cross-sectional view of the nozzle 108 shown in FIGS. 2 and 4, the nozzle 108 has a base portion 136 selectively mounted directly to the container and a top face 138 spaced from the base portion 136 with the top face 138 defining a discharge opening 140. The nozzle 108 has an interior 142 extending between the base portion 136 and the top face 138 and a rim 144 disposed between the base portion 136 and the top face 138 and projecting radially outward relative to the central axis C, and a side wall 146 extending between the top face 138 and the rim 144 with the side wall 146 being continuous and uninterrupted between the top face 138 and the rim 144 for conforming to the tissue surrounding the orifice to seal the orifice during delivery of the fluid from the container into the human cavity or the animal cavity. The nozzle 108 may also include a flexible membrane 148 disposed within the nozzle 108 and adjacent to the rim 144 to prevent fluid from being ejected prior to positioning and to prevent back flow into the container. The base portion 136 has a circumferential outer surface that extends downwardly to define a barrier region 150. The barrier region 150 is adjacent to the abutment flange 128 when the nozzle 108 is connected to the container.

Once the anal opening is located, the device 100 can be pressed gently against the anus whereby the tissue surrounding the anal opening will conform to the non-penetrating nozzle 108 creating a seal that will make the anal opening the path of least resistance for the fluid when the bottle is squeezed. The device 100 has a safety flex neck that simultaneously provides a seal and convenience for the user. By maintaining the non-penetrating nozzle's gentle pressure against the anus while squeezing the bottle, the area around the anal opening will remain conformed to the non-penetrating nozzle 108 maintaining the necessary seal that will allow the water to jettison into the anal opening past the exterior and interior sphincter muscles and into the rectal cavity. Fluid will be delivered to the same destination as anal-penetrating nozzle 108 enemas without the common penetration discomfort and without the danger of injuring delicate rectal tissue.

The invention has been described in an illustrative manner, and it is to be understood that the terminology which has been used is intended to be in the nature of words of description rather than of limitation. As is now apparent to those skilled in the art, many modifications and variations of the present invention are possible in light of the above teachings. It is, therefore, to be understood that within the scope of the appended claims, wherein reference numerals are merely for convenience and are not to be in any way limiting, the invention may be practiced otherwise than as specifically described.

What is claimed is:

1. A device for administering a fluid into a human cavity or animal cavity through an orifice, said device comprising:
a container for housing the fluid;
wherein said container comprises a bottom portion, a tip portion, and a directional flange disposed between said bottom portion and said tip portion;
said bottom portion having a bottom, a squeezing section, and a narrowing section adjacent said directional flange;
said tip portion having an end section with an end face and a body section defining an elongated structure extending from said directional flange to said end face; and
wherein said directional flange comprises an upper surface and a lower surface connected to one another by an edge and said edge has a rigidity less than said tip portion such that said edge collapses in response to a force applied to said tip portion causing movement of said end face relative to said bottom portion.

2. A device as set forth in claim 1 wherein said upper surface is further defined as having an inclined upper angle greater than zero degrees and less than 55 degrees.

3. A device as set forth in claim 1 wherein said lower surface is further defined as a recessed surface with an inclined lower angle of less than 20 degrees.

4. A device as set forth in claim 1 wherein said lower surface is further defined as a substantially flat surface with an inclined lower angle of less than 3 degrees.

5. A device as set forth in claim 1 wherein said directional flange has a first height when no forces are applied to said tip portion and a second height when an orthogonal force is applied to said tip portion.

6. A device as set forth in claim 1 wherein said directional flange has a first height when no forces are applied to said tip portion and a compressed height and a stretched height when a transverse force is applied to said tip portion.

7. A device as set forth in claim 1 wherein said narrowing section of said bottom portion has a rigidity more than said edge for transmitting forces to said directional flange and allowing movement therebetween.

8. A device as set forth in claim 1 wherein said upper and lower surfaces have a rigidity that is greater than said rigidity of said edge.

9. A device as set forth in claim 1 wherein said edge is further defined as having a wall thickness less than a wall thickness of said body section of said tip portion for allowing movement therebetween when said tip portion is subjected to a force.

10. A device as set forth in claim 9 wherein said upper and lower surfaces have a wall thickness that is the same as said wall thickness of said edge.

11. A device as set forth in claim 9 wherein said upper surface has a wall thickness that is greater than as said wall thickness of said edge.

12. A device as set forth in claim 9 wherein said lower surface has a wall thickness that is greater than as said wall thickness of said edge.

13. A device as set forth in claim 1 wherein said upper surface and said lower surface have a wall thickness that is the same as each other.

14. A device as set forth in claim 1 wherein said tip portion is further defined as having a conical shape and tapering towards said end face.

15. A device as set forth in claim 14 further comprising a nozzle coupled to said container for delivering the fluid from said container into the human cavity or the animal cavity through the orifice.

16. A device as set forth in claim 15 wherein said tip portion further comprises threads for having a threaded connection with said nozzle.

17. A device as set forth in claim 16 wherein said nozzle has a base portion selectively mounted to said container, a top face spaced from said base portion with said top face defining a discharge opening, an interior extending between said base portion and said top face, a rim disposed between said base portion and said top face and projecting radially outward relative to a vertical axis, and a side wall extending between said top face and said rim with said side wall being continuous and uninterrupted between said top face and said rim for conforming to a tissue surrounding the orifice to seal the orifice during delivery of the fluid from said container into the human cavity or the animal cavity.

18. A device as set forth in claim 17 further comprising a flexible membrane disposed within said nozzle and adjacent to said rim to prevent fluid from being ejected prior to positioning and to prevent back flow into said container.

19. A device as set forth in claim 17 wherein said tip portion further comprises an abutment flange and wherein said base portion is adjacent to said abutment flange when said nozzle is connected to said container.

20. A device as set forth in claim 1 wherein said bottom is further defined as having a partial flat surface for supporting said device.
